(19) United States
(12) Patent Application Publication
(10) Pub. No.: US 2021/0002646 A1
(43) Pub. Date: Jan. 7, 2021

(54) DNMT3B GENE-DEFICIENT CHO CELL LINE, PREPARATION AND APPLICATIONS THEREOF AND RECOMBINANT PROTEIN EXPRESSION SYSTEM USING THE SAME

(71) Applicants: Xinxiang Medical University, Xinxiang (CN); Proteineasy Biological Products Research Institute (Henan) Co., Ltd, Xinxiang (CN)

(72) Inventors: Yanlong JIA, Xinxiang (CN); Tianyun WANG, Xinxiang (CN); Jiangtao LU, Xinxiang (CN); Xiao GUO, Xinxiang (CN); Tianjun NI, Xinxiang (CN); Mengke XIAO, Xinxiang (CN); Lele QIU, Xinxiang (CN); Yan LIN, Xinxiang (CN); Chunpeng ZHAO, Xinxiang (CN)

(21) Appl. No.: 16/862,664

(22) Filed: Apr. 30, 2020

(30) Foreign Application Priority Data
Jul. 2, 2019 (CN) 201910590913.6

(51) Int. Cl. C12N 15/113; C12N 9/22; C12N 15/85

(52) U.S. Cl. CPC C12N 15/1137; C12N 9/22; C12N 15/85; C12N 2810/10; C12N 2800/107; C12N 2800/80; C12N 2310/20

(57) ABSTRACT

The invention relates to genetic engineering, and more particularly to a Dnmt3b gene-deficient CHO cell line, a preparation method and an application thereof and a recombinant protein expression system using the same. The invention adopts a CRISPR/Cas9 gene editing technique to knock out the Dnmt3b gene from the CHO cells to produce the Dnmt3b gene-deficient CHO cell line, which can significantly improve the expression level and stability of the target gene in CHO cells, overcoming the defects existing in the current CHO cell expression system, such as low expression level and stability. It has been demonstrated that using the CHO line provided herein to express a recombinant adalimumab can significantly increase the expression level of the recombinant adalimumab, indicating that the CHO cell line can be widely used to enhance the expression of target proteins.

### REFERENCE TO AN ELECTRONIC SEQUENCE LISTING

The contents of the electronic sequence listing (Untitled ST25.txt; Size: 36,000 bytes; and Date of Creation: Aug. 14, 2020) is herein incorporated by reference in its entirety.

### CROSS-REFERENCE TO RELATED APPLICATIONS

This application claims the benefit of priority from Chinese Patent Application No. 201910590913.6, filed on Jul. 2, 2019. The content of the aforementioned application, including any intervening amendments, reported herein by reference in its entirety.

### TECHNICAL FIELD

This application relates to genetic engineering, and more particularly to a Dnmt3b gene-deficient CHO cell line, a preparation method and applications thereof and a recombinant protein expression system using the same.

### BACKGROUND

Due to the outstanding ability to perform the assembly and folding of recombinant proteins and the post-translational modification, mammalian cells, especially Chinese hamster ovary (CHO) cells, are often applied in the expression and production of recombinant pharmaceutical proteins. However, there are some defects currently in the long-term culture and production of recombinant CHO cells, such as low expression, unstable expression and even significantly-reduced expression. In this regard, there is an urgent need to seek for a method to improve the expression level and expression stability in mammalian cells in the large-scale production of recombinant proteins. Currently, due to the lack of a recombinant CHO cell line capable of ensuring both high yield and stable expression, the production of recombinant proteins is still greatly limited in scale. Considering the above, using molecular cloning and genetic engineering to construct a recombinant CHO cell expression system is of great theoretical and practical significance for improving the expression level and long-term expression stability of recombinant proteins, and further establishing a high-yield and stable recombinant CHO cell expression system.

The exact mechanism responsible for the unstable expression of this recombinant protein still has not been fully demonstrated. The decrease in the expression of transgenes is closely associated with the reduction of transcripts and the occurrence of transcriptional silencing which are caused by the gradual loss of gene copies during continuous expression. With the help of some epigenetic-regulating DNA cis-acting elements such as matrix attachment region (MAR) sequence and ubiquitous chromatin open element (UCOE), the gene silencing can be significantly reduced to promote the expression of the recombinant target gene. In addition, the transgene silencing is closely related to the methylation of the promoter CpG site on the recombinant protein expression vector which drives gene expression during production. For example, it has been found that in recombinant CHO cells with unstable production and expression of monoclonal antibody, the methylation level of the promoter is increased in the human cytomegalovirus-main immediate early stage (hCMV-MIE), and after treated with 5-Aza-2-deoxycytidine (a DNA methylation inhibitor), these cells with reduced expression can be partially recovered with regard to the yield of monoclonal antibody. Such results indicate that epigenetic factors, such as DNA methylation, play an important role in regulating gene expression, and the abnormal increase in the methylation of DNA in the promoter region may be related to the reduction in the expression of target gene in the recombinant CHO cells.

DNA methylation, as a primary epigenetic modification, commonly occurs in genomes of prokaryotes and eukaryotes, and also plays an important role in regulating gene expression and inhibiting gene transcription in mammals. Currently, researches on the effect of DNA methylation on the transgene expression in CHO cells mainly focus on the modification for the promoter, such as the mutation of cytosine in the promoter region, the use of a CpG-free or synthetic promoter and the insertion of a core CpG island into the promoter. Though these CpG dinucleotide-free promoters can reduce the early gene silencing, they still fail to significantly improve the long-term expression stability of recombinant CHO cells. The DNA methylation is mainly performed under the catalytic action of DNA methyltransferases including de novo methyltransferases Dnmt3a and Dnmt3b and DNA methyltransferase Dnmt1, where the Dnmt3a- and Dnmt3b-mediated promoter methylation is closely related to the unstable expression of transgenes, which indicates that these two methyltransferases play an important role in regulating the stability of gene expression.

Chinese Patent Application Publication No. 107828738A discloses a DNA methyltransferase-deficient CHO cell line, which can significantly increase the expression level of recombinant protein EPO and overcome the problem of unstable expression of the recombinant protein, greatly improving the expression stability of the recombinant protein. However, this system was still found to have some defects through the verification of expression stability, for example, the expression stability of the target protein is unsatisfactory; and a certain degree of DNA methylation still occurs to the promoter. In addition, in the absence of G418 screening, the expression maintenance rate of recombinant proteins still fails to reach 70% after 30 passages, which indicates a relatively low expression level according to the requirements of the long-term expression without pressure screening in production.

### SUMMARY

A first object of the invention is to provide a DNA methyltransferase (Dnmt3b) gene-deficient CHO cell line, where the Dnmt3b-mediated epigenetic modification is more closely related to the regulation of gene expression. The deletion of Dnmt3b gene will facilitate improving the expression stability and expression level of a heterologous protein in the absence of screening pressure.

A second object of the invention is to provide a method of constructing the above-mentioned Dnmt3b gene-deficient CHO cell line, which has a high success rate.

A third object of the invention is to provide an application of the above-mentioned Dnmt3b gene-deficient CHO cell line in the efficient expression of target proteins.

A forth object of the invention is to provide a recombinant protein expression system using the above-mentioned Dnmt3b gene-deficient CHO cell line, which facilitates the efficient expression of the target proteins.

The technical solutions of the invention are described as follows.

In a first aspect, the invention provides a Dnmt3b gene-deficient CHO cell line, wherein the Dnmt3b gene in the Dnmt3b gene-deficient CHO cell line is lost in function, and the Dnmt3b gene has a sequence as shown in SEQ ID NO: 1.

The Dnmt3b gene-deficient CHO cell line provided herein can significantly improve the expression of target genes in level and stability in CHO cells when used to express target target proteins, overcoming the defects existing in the current CHO cell expression system, such as low expression level and stability. Since the epigenetic modification mediated by the DNA methyltransferase Dnmt3b gene is more closely related to the regulation of gene expression, the CHO cell line of the invention in which the Dnmt3b gene is deleted can ensure more stable expression of the target gene. In the absence of screening pressure, the expression level of the recombinant protein eGFP in the cells of passage 30 is greater than 70% of that in the CHO cells of passage 1, which demonstrates that the expression level of the target gene is still relatively high in the CHO cells after 30 passages.

In an embodiment, the Dnmt3b gene-deficient CHO cell line is prepared from a CHO cell line selected from the group consisting of CHO-K1, CHO-S and CHO-DG44.

In an embodiment, various commercially-available CHO cell lines can be used to prepare the above-mentioned Dnmt3b gene-deficient CHO cell line.

In a second aspect, the invention provides a method of preparing the Dnmt3b gene-deficient CHO cell line of claim 1, comprising:

knocking out the Dnmt3b gene from CHO cells by CRISPR/Cas9 gene editing technique to produce the Dnmt3b gene-deficient CHO cell line.

The invention adopts the CRISPR/Cas9 gene editing technique to knock out the Dnmt3b gene from the CHO cells, which provides high knockout efficiency.

In an embodiment, in the method, the step of “knocking out the Dnmt3b gene from CHO cells” comprises:

(1) designing sgRNA sequences I and II as target sites according to the sequence of the Dnmt3b gene;

(2) adding a first sticky end and a second sticky end respectively to the sgRNA sequences I and II; synthesizing two pairs of primers from the sgRNA sequences I and II; subjecting the two pairs of primers to annealing to correspondingly produce double-stranded DNA fragments; and respectively ligating the double-stranded DNA fragments into two CRISPR/Cas9 expression vectors respectively carrying fluorescent reporter genes I and II to construct two CRISPR/Cas9-sgRNA vectors; and

(3) co-transfecting the two CRISPR/Cas9-sgRNA vectors into the CHO cells; selecting monoclonal cells containing the two fluorescent reporter genes I and II by flow cytometry for culture; and subjecting the monoclonal cells to knockout verification to obtain the Dnmt3b gene-deficient CHO cell line.

Two sgRNA sequences are designed herein as target sites, so that the relatively-large sequence between the two target sites is deleted during the knockout of the Dnmt3b gene, ensuring the complete loss of the functions of the Dnmt3b gene.

In an embodiment, in step (1), the two sgRNA sequences are respectively shown as follows:

D3b-Ex1-31fw:
(SEQ ID NO: 5)
5′-GAGGAATGTCTCATCGTCAATGG-3′;
and
D3b-Ex1-105fw:
(SEQ ID NO: 6)
5′-CTTGGAGGCAATGTGCACAGAGG-3′.

The two sgRNA sequences designed herein as target sites can ensure a highly-efficient knockout of the Dnmt3b gene.

In an embodiment, in step (2), the two pairs of primers are respectively shown as follows:

D3b-Ex1-31fw-1:
(SEQ ID NO: 7)
5′-CACCGAGGAATGTCTCATCGTCAATGG-3′;
D3b-Ex1-31fw-2:
(SEQ ID NO: 8)
5′-AAACCCATTGACGATGAGACATTCCTC-3′;
D3b-Ex1-105fw-3:
(SEQ ID NO: 9)
5′--CACCGCTTGGAGGCAATGTGCACAGAGG-3′;
and
D3b-Ex1-105fw-4:
(SEQ ID NO: 10)
5′-AAACCCTCTGTGCACATTGCCTCCAAGC-3′.

The two pairs of primers are designed respectively based on the two sgRNA sequences.

In an embodiment, in step (3), primers employed in the knockout verification are shown as follows:

Dnmt3b-Ex1PCR-L:
(SEQ ID NO: 3)
5′-GTGCCCCCATTTCTCCTACT-3′;
and
Dnmt3b-Ex1PCR-R:
(SEQ ID NO: 4)
5′-AGACCCAATGTGCTGGTCTC-3′.

This pair of primers can be used to synthesize a fragment with a length of 288 bp by amplification. During the verification, if the amplified fragment has a length significantly less than 288 bp, it indicates that the Dnmt3b gene is knocked out in the cell line.

In a third aspect, the invention provides a use of the Dnmt3b gene-deficient CHO cell line in the preparation of a target protein.

The Dnmt3b gene-deficient CHO cell line provided herein can significantly improve the expression level and stability of the target gene in the CHO cells, overcoming the defects of low expression level and stability in the current CHO cell expression system. Therefore, the Dnmt3b gene-deficient CHO cell line facilitates the expression of target proteins.

In a forth aspect, the invention further provides a recombinant protein expression system, which is prepared by the steps of:

inserting a target gene into an expression vector to construct a recombinant protein expression vector; and transfecting the recombinant protein expression vector into the Dnmt3b gene-deficient CHO cell line followed by screening to obtain the recombinant protein expression system; wherein the recombinant protein expression system is capable of expressing a target protein corresponding to the target gene.

The recombinant protein expression system of the invention is constructed based on the Dnmt3b gene-deficient CHO cell line, and this system is capable of significantly improving the expression level and stability of the target gene in the CHO cells, overcoming the defects of low expression level and stability in the current CHO cell expression system.

In an embodiment, the target protein is a recombinant adalimumab.

The recombinant protein expression system of the invention can significantly increase the expression level of the recombinant adalimumab.

### BRIEF DESCRIPTION OF THE DRAWINGS

FIG. 1 shows the screening of a Dnmt3b gene-knockout CHO monoclonal cell line of the invention by PCR amplification.

FIG. 2 shows the detection of the proliferation of Dnmt3b gene-deficient CHO monoclonal cell lines 3b-2 and 3b-7 and normal CHO-K1 cells.

FIG. 3 shows the detection of apoptosis of Dnmt3b-deficient CHO cells and normal CHO cells by flow cytometry.

FIG. 4a is a fluorescence image showing the transient expression of eGFP gene driven by the CMV promoter in Dnmt3b-deficient CHO cells;

FIG. 4b is a white light image showing the transient expression of eGFP gene driven by the CMV promoter in Dnmt3b-deficient CHO cells;

FIG. 4c is a fluorescence image showing the transient expression of eGFP gene driven by the CMV promoter in normal CHO-K1 cells; and

FIG. 4d is a white light image showing the transient expression of eGFP gene driven by the CMV promoter in normal CHO-K1 cells.

FIG. 5 shows the expression stability of eGFP gene driven by the CMV promoter in Dnmt3b-deficient CHO cells (3b-7) and normal CHO-K1 cells.

FIG. 6 shows Western Blot results of recombinant adalimumab in Dnmt3b-deficient CHO cells (3b-7) and normal CHO cells.

### DETAILED DESCRIPTION OF EMBODIMENTS

The invention will be described in detail below with reference to the embodiments, and these embodiments are not intended to limit the invention. Unless otherwise specified, instruments and reagents used in the following examples and experimental examples are all commercially available.

### Example 1 Preparation of Methyltransferase Dnmt3b Gene-Deficient CHO Cell Line

Provided herein was a method of preparing a DNA methyltransferase Dnmt3b gene-deficient CHO cell line, which was specifically described as follows.

1. Determination of Target Sites for a Candidate Gene

(1) Partial Amplification of Sequence of DNA Methyltransferase Dnmt3b Gene

Primers for amplification were designed according to the sequence of the Chinese hamster DNA methyltransferase Dnmt3bgene (No. NW_006879210) recorded in GenBank of NCBI, and were shown as follows:

Dnmt3b-Ex1PCR-L:
(SEQ ID NO: 3)
5′-GTGCCCCCATTTCTCCTACT-3′;
and
Dnmt3b-Ex1PCR-R:
(SEQ ID NO: 4)
5′-AGACCCAATGTGCTGGTCTC-3′.

The Dnmt3b gene fragment was amplified by PCR, and the amplified product was cloned and sequenced for verification. The desired amplified sequence was shown in SEQ ID NO: 2.

(2) Determination of sgRNA Sequences of Target Sites

The sgRNA sequences of target sites for the Dnmt3b gene were designed with the help of an online tool (http://crispr.mit.edu/), and were shown as follows:

D3b-Ex1-31fw:
(SEQ ID NO: 5)
5′-GAGGAATGTCTCATCGTCAATGG-3′;
and
D3b-Ex1-105fw:
(SEQ ID NO: 6)
5′-CTTGGAGGCAATGTGCACAGAGG-3′.

2. Construction of a sgRNA Expression Vector

(1) Designing and Synthesis of Primers

Two pairs of primers were designed and synthesized according to the above sgRNA sequences of target sites, and respectively added with a sticky end at the 5′ end. The primers were shown as follows:

D3b-Ex1-31fw-1:
(SEQ ID NO: 7)
5′-CACCGAGGAATGTCTCATCGTCAATGG-3′;
D3b-Ex1-31fw-2:
(SEQ ID NO: 8)
5′-AAACCCATTGACGATGAGACATTCCTC-3′;
D3b-Ex1-105fw-3:
(SEQ ID NO: 9)
5′-CACCGCTTGGAGGCAATGTGCACAGAGG-3′;
and
D3b-Ex1-105fw-4:
(SEQ ID NO: 10)
5′-AAACCCTCTGTGCACATTGCCTCCAAGC-3′.

Since a U6 promoter was used in the sgRNA expression vector, the gene expression will be significantly up-regulated in the presence of a guanine (G) in the starting site of the gene transcription. Therefore, during the designing process of the primers, if the starting base at 5′ end of the forward primer was G, it was required to additionally add a guanine to ensure a high expression level. In this case, a cytosine (C) was required to be added at the 3′ end of the corresponding reverse primer.

(2) Preparation of Double-Stranded DNA Fragments by Annealing

The two pairs of primers (D3b-Ex1-31fw-1+D3b-Ex1-31fw-2; D3b-Ex1-105fw-3+D3b-Ex1-105fw-4) obtained in step (1) were respectively subjected to annealing to produce double-stranded DNA fragments both with a sticky end. Specifically, the two pairs of primers were respectively phosphorylated and then transferred to a PCR instrument for denaturation and annealing, where the phosphorylation was performed through the steps of: mixing 1.0 μL of respective primers (100 μM), 1.0 μL of 10×T4 Ligation Buffer (NEB), 0.5 μL of T4Polynucleotide Kinase (NEB M0201S) and 6.5 μL of ddH₂O uniformly to produce a phosphorylation system (10 μL); and incubating the phosphorylation system at 37° C. for 30 min to complete the phosphorylation; the denaturation was performed at 95° C. for 5 min; and the annealing was performed by reducing the temperature from 95° C. to 25° C. at 5° C./min.

(3) Linearization of CRISPR/Cas9 Expression Vectors

Two CRISPR/Cas9 expression vectors pX458-ECFP carrying the gene of fluorescent protein ECFP and pX458-DsRed2 carrying the gene of fluorescent protein DsRed2 were linearized in the presence of endonucleaseBbs I, purified and recovered to obtain DNA fragments of the vectors, where the digestion was performed through the steps of: mixing 1.0 μg of the pX458-DsRed2 or pX458-ECFP vector, 3.0 μL of 10×NEB Buffer 2.1 and 1.0 μL of Bbs I (NEB) followed by addition of ddH₂O to a volume of 30.0 μL; and incubating the digestion system at 37° C. for 2 h to complete the digestion; and the digested product was purified using a QIAquick PCR Purification Kit and dissolved with 30.0 μL of ddH₂O for recovery.

(4) Construction of sgRNA Expression Vectors

CRISPR/Cas9 expression vectors containing sgRNA were obtained by ligation, transformation and screening, where the ligation was performed through the steps of mixing 0.5 μL of the double-stranded DNA fragment with the sticky end obtained in step (2), 2.0 μL of the vector DNA with the same sticky end obtained in step (3), 0.5 μL of T4DNA ligase (NEB M0202S) and 1.0 μL of 10×T4 Ligation Buffer (NEB) followed by addition of ddH₂O to a volume of 10.0 μL to produce a ligation system; and reacting the ligation system for 1 h to complete the ligation; the transformation and screening were performed through the steps of transforming the ligated product into E. coli DH5a cells; spreading the cells on a ampicillin-resistant plate; incubating the plate at 37° C. overnight; and picking up a single colony for sequencing verification to obtain the expression vectors pX458-3b-1 and pX458-3b-2 respectively capable of expressing ECFP and DsRed2.

3. Transfection of CHO Cells, and Screening and Identification of Gene-Knockout Monoclonal Cell Line

The pX458-3b-1 and pX458-3b-2 expression vectors were mixed in equal weight and transfected into CHO cells in a liposome-mediated manner. Then the CHO cells were screened to obtain Dnmt3b gene-knockout monoclonal cell line.

The above process was specifically described as follows. CHO-K1 cells were cultured in a DMEM medium containing 10% inactivated fetal bovine serum at 37° C. and 5% CO₂. Before the transfection, 2.0×10⁵ CHO-K1 cells were seeded in a 24-well culture plate and cultured for 24 h. When the confluency reached about 90%, the cells were used for the transfection. The pX458-3b-1 and pX458-3b-2 vectors each for 1.5 g were diluted with 150.0 μL of a reduced serum media (Opti-MEM). 0.75 μL of a liposome Lipofectamine 3000 was diluted with 150.0 μL of the reduced serum media (Opti-MEM) and added to the diluted expression vector medium. Then the reaction mixture was fully mixed and incubated at room temperature for 20 min. The medium in the 24-well plate was discarded, and then the CHO-K1 cells in respective wells were added with 300 μL of the mixture of the vector and the liposome. Another three wells were treated in the same manner and used as parallel controls, and the wells in which the cells were not added with the transfection mixture were used as negative control. All cells were cultured at 37° C. and 5% CO₂ for 1.5 h, and then the medium was replaced for continuous culture.

In order to obtain stable Dnmt3b gene-knockout monoclonal cells, single cells with double positive of DsRed2 and ECFP were sorted by flow cytometry into a 96-well plate containing 150 μL of fresh medium after 72 h of the transfection, and cultured for 14 d. After that, the resulting monoclonal cells were transferred to a 48-well plate for enlarged culture and subsequent PCR verification.

The extraction of genomic DNA from monoclonal cells was performed as follows. A small number of cells (about 1×10⁶-10⁷) were collected and centrifuged at 350×g for 5 min. The supernatant was discarded, and the cells were added with 20.0 μL of a cell lysis solution containing 100 mM KCl, 20 mM Tris-HCl (pH 9.0), 0.3% Triton X-100 and 1.0 mg/mL proteinase K, gently mixed using a pipette and incubated at 55° C. for 15 min for complete lysis. Then the system was incubated at 95° C. for 10 min to denature the proteinase K, and the resulting lysate containing the genomic DNA of the cells can be used as a PCR template and stored at −20° C. for use.

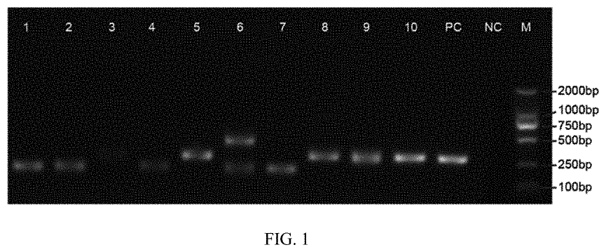

Primers Dnmt3b-Ex1PCR-L and Dnmt3b-Ex1PCR-R were used to amplify the target fragment containing the target site by PCR, and sequencing analysis was performed to determine whether the base deletion or insertion occurred in the monoclonal cell lines. Results of the agarose gel electrophoresis detection for PCR products were shown in FIG. 1, where PC indicated positive plasmid control; NC indicated blank negative control; and M indicated DNA molecular weight marker. It can be seen from FIG. 1 that single-cell clones 1, 2, 4, and 7 were homozygotes with deletion of a large fragment. The single-cell clones 1, 2, 4, and 7 were further identified by sequencing analysis to be Dnmt3b gene-knockout cell lines. Then these Dnmt3b gene-knockout monoclonal cell lines were subjected to enlarged culture and cryopreserved in liquid nitrogen.

4. Identification of Biological Characteristics of Dnmt3b-Deficient CHO Cells

Whether the Dnmt3b gene-deficient cell line can perform normal growth and passage was demonstrated by examining biological characteristics of the cells such as cell proliferation and apoptosis.

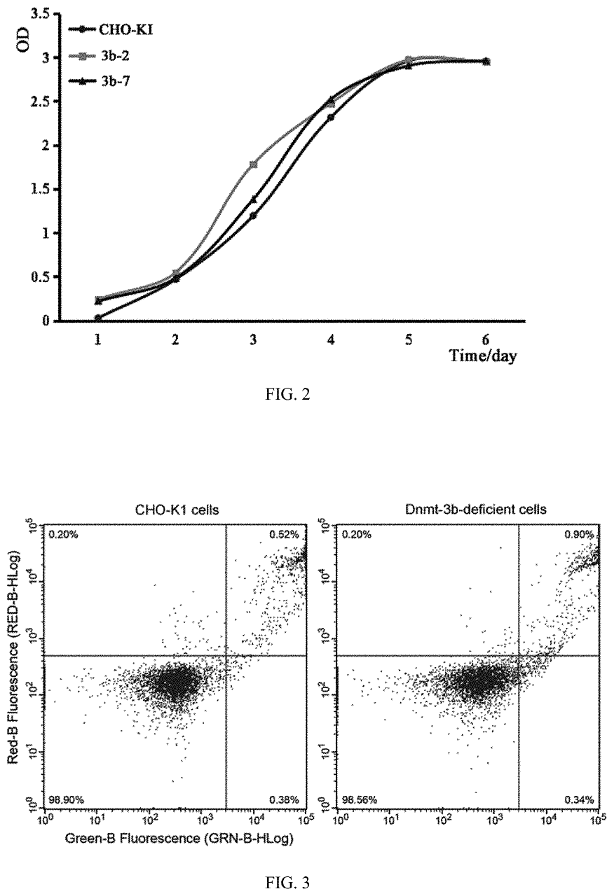

Wild CHO-K1 cells were used as control to verify the growth characteristics of the obtained Dnmt3b-deficient CHO monoclonal cells, determining whether the Dnmt gene-deficient CHO cell line can perform normal growth and passage. Specifically, the verification included observation of cell morphology and growth status, detection of cell proliferation by CCK-8 assay and the examination of cell apoptosis by flow cytometry (FCM), where Cell Counting Kit-8 (CCK-8) was employed; the results of the detection of cell proliferation were shown in FIG. 2; and the results of the examination of cell apoptosis were shown in FIG. 3.

It can be seen from the results that there was no significant difference between the Dnmt3b-deficient CHO cell line and the normal CHO cells in the biological characteristics such as growth status, morphology, proliferation and apoptosis, indicating that the Dnmt3b-deficient CHO cell line can perform normal growth, proliferation and passage.

### Example 2 Application of the Dnmt3b Gene-Deficient CHO Cell Line

Expression of Target Gene eGFP in Recombinant CHO Cells

1. Transfection of CHO Cells

Dnmt3b gene-deficient CHO cells (3b-7) and normal CHO cells (CHO-K1) were used herein. These two types of CHO cells were cultured, and inoculated into a fresh DMEM medium containing 10% inactivated fetal bovine serum at a density of 2×10⁵ cells one day before the transfection. When the conflency reached 90%, Lipofectamine 3000 was used to perform the transfection, where the plasmid used herein was an eukaryotic expression vector pWTY-02 constructed by our laboratory, in which the expression of enhanced green fluorescent protein (eGFP) was driven by the CMV promoter. The transfection was performed in three parallel replicates.

2. Transient Expression of Transfected Cell Lines

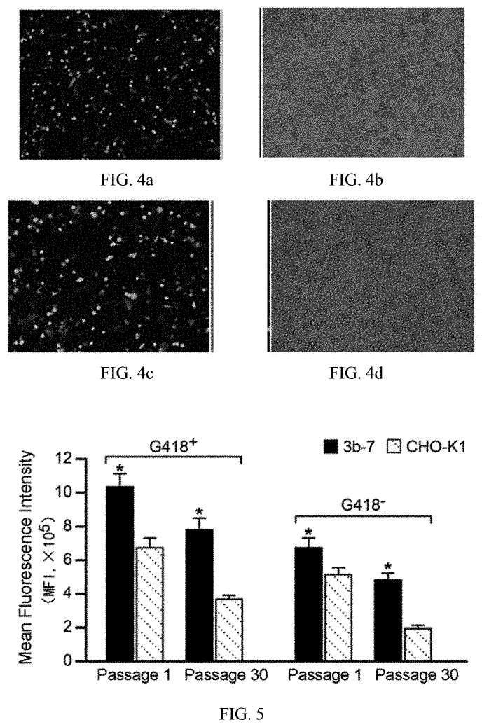

48 h after the transfection, the transient expression of eGFP in the two groups of cells was observed by an inverted fluorescence microscope, and the results were shown in FIGS. 4A-4D, where FIG. 4A was a fluorescence image showing the transient expression of eGFP gene in Dnmt3b-deficient CHO cells; FIG. 4B was a white light image showing the transient expression of eGFP gene in Dnmt3b-deficient CHO cells; FIG. 4C was a fluorescence image showing the transient expression of eGFP gene in normal CHO-K1 cells; and FIG. 4D was a white light image showing the transient expression of eGFP gene in normal CHO-K1 cells.

It can be concluded from the results that the expression vector had comparable transfection efficiency in Dnmt 3b-deficient and normal CHO-K1 cells, and there was no significant difference in the ratio of the eGFP-positive cells between the two groups, which indicated that the knockout of Dnmt 3b gene showed no significant effect on the transfection efficiency of cells.

3. Screening of a Polyclonal CHO Cell Line with Stable Expression and Analysis of Long-Term Stable Expression of eGFP

The cells were screened in the presence of G418, and stably-transformed polyclonal cell pools were obtained after two weeks of screening. Then the cell pools were cultured to passage 30 respectively in the presence (G418+) and absence (G418-) of G418, and 10⁶ CHO cells from respective groups were analyzed by flow cytometry to measure the mean fluorescence intensity (MFI) of eGFP. It can be seen from the results that according to the criterion for evaluating the expression stability (whether the expression level of eGFP in CHO cells at passage 30 was greater than 70% of that in CHO cells at passage 1), the expression stability of eGFP whose expression was driven by the CMV promoter in recombinant Dnmt3b-deficient CHO cells was significantly higher that in the normal CHO cells (see FIG. 5).

### Example 3 Another Application of the Dnmt3b Gene-Deficient CHO Cell Line

Expression of Recombinant Antibody (Adalimumab) in Recombinant CHO Cells

An eukaryotic expression vector, in which the expression of adalimumab was driven by the CMV promoter, was constructed based on the plasmid pWTY-02 to further test the effect of Dnmt3b-deficient CHO cells on the expression stability of recombinant protein (antibody). The expression vector plasmid was respectively transfected into Dnmt3b-deficient cells (3b-7) and normal CHO cells (CHO-K1), and then the transfected cells were cultured in a medium containing 800 μg/mL of G418 for 15 days to screen stably-transfected recombinant cell pools. The recombinant CHO cells were passaged every other 3 days and cultured to passage 30. Subsequently, the recombinant CHO cells were cultured in a 125 mL shaking flask containing 30 mL of a protein-free, serum-free, chemically-defined (CD) CHO medium with 8 mM L-glutamine (Life Technologies Co., Ltd.) for 6 days to the cell number of 1.5×10⁷, where the cells were collected every day and detected by a Countstar® BioTech cell counter (Shanghai Ruiyu Biotechnology Co., Ltd.) for the density and viability, and the supernatant was collected by centrifugation on the 6th day and then analyzed for the expression of the recombinant adalimumab.

The expression of adalimumab in the recombinant Dnmt3b-deficient cells (3b-7) and normal CHO cells (CHO-K1) was analyzed by Western Blot. Specifically, the supernatant containing adalimumab was added to a 5×SDS sample buffer and boiled in a water bath for 10 min for complete denaturation. 25 μL of the denatured protein sample was separated by 10% SDS-polyacrylamide gel electrophoresis and then transferred to a nitrocellulose membrane by a wet transfer method. The membrane was blocked with 5% BSA for 2 h, incubated with goat anti-human secondary antibody diluted by 1:6000 at room temperature for 1.5 h, washed three times with TBST and developed with a chemiluminescent detection reagent. The image was observed and collected using a gel imager to analyze the gray value of the target protein, obtaining the expression level of the protein to be tested.

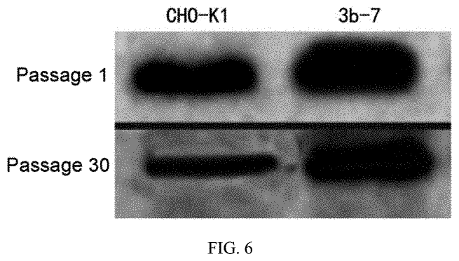

It can be seen from the results that the expression level of the recombinant adalimumab in Dnmt3b-deficient CHO cell pool (1217.1±49.9 mg/L) was significantly higher than that in the normal CHO cell pool (392.6±37.3 mg/L). Moreover, after cultured to passage 30, the expression levels of the recombinant adalimumab in Dnmt3b-deficient CHO cell pool and normal CHO cell pool were respectively 985.8±58.5 mg/L and 160.9±26.6 mg/mL, indicating that the difference in expression level was of statistical significance (P<0.05, see FIG. 6). These results demonstrated that the expression and stability of recombinant adalimumab in Dnmt3b-deficient CHO cells were significantly improved.

### Example 4 Preparation of Recombinant Protein Expression System

The recombinant protein expression system provided herein was prepared as follows. Specifically, the target gene was inserted into an expression vector to construct a recombinant protein expression vector. Then the recombinant protein expression vector was transfected into the above Dnmt3b-deficient CHO cell line and screened to obtain the recombinant protein expression system capable of expressing the target protein.

What is claimed is:

1. A Dnmt3b gene-deficient CHO cell line, wherein the Dnmt3b gene in the Dnmt3b gene-deficient CHO cell line is lost in function, and the Dnmt3b gene has a sequence as shown in SEQ ID NO: 1.

2. The Dnmt3b gene-deficient CHO cell line of claim 1, wherein the Dnmt3b gene-deficient CHO cell line is derived from a CHO cell line selected from the group consisting of CHO-K1, CHO-S and CHO-DG44.

3. A method of preparing the Dnmt3b gene-deficient CHO cell line of claim 1, comprising:
knocking out the Dnmt3b gene from CHO cells by using a CRISPR/Cas9 gene editing technique to produce the Dnmt3b gene-deficient CHO cell line.

4. The method of claim 3, wherein the step of “knocking out the Dnmt3b gene from CHO cells” comprises:
(1) designing sgRNA sequences I and II as target sites according to the sequence of the Dnmt3b gene;
(2) adding a first sticky end and a second sticky end respectively to the sgRNA sequences I and II; synthesizing two pairs of primers from the sgRNA sequences I and II; subjecting the two pairs of primers to annealing to correspondingly produce double-stranded DNA fragments; and respectively ligating the double-stranded DNA fragments into two CRISPR/Cas9 expression vectors respectively carrying fluorescent reporter genes I and II to construct two CRISPR/Cas9-sgRNA vectors; and
(3) co-transfecting the two CRISPR/Cas9-sgRNA vectors into the CHO cells;
selecting monoclonal cells containing the two fluorescent reporter genes I and II by flow cytometry for culture; and subjecting the monoclonal cells to knockout verification to obtain the Dnmt3b gene-deficient CHO cell line.

5. The method of claim 4, wherein in step (1), the sgRNA sequences I and II are respectively shown as follows:
D3b-Ex1-31fw:
(SEQ ID NO: 5)
5′-GAGGAATGTCTCATCGTCAATGG-3′;
and
D3b-Ex1-105fw:
(SEQ ID NO: 6)
5′-CTTGGAGGCAATGTGCACAGAGG-3′.

6. The method of claim 5, wherein in step (2), the two pairs of primers are respectively shown as follows:
D3b-Ex1-31fw-1:
(SEQ ID NO: 7)
5′-CACCGAGGAATGTCTCATCGTCAATGG-3′;
D3b-Ex1-31fw-2:
(SEQ ID NO: 8)
5′-AAACCCATTGACGATGAGACATTCCTC-3′;
D3b-Ex1-105fw-3:
(SEQ ID NO: 9)
5′--CACCGCTTGGAGGCAATGTGCACAGAGG-3′;
and
D3b-Ex1-105fw-4:
(SEQ ID NO: 10)
5′-AAACCCTCTGTGCACATTGCCTCCAAGC-3′.

7. The method of claim 5, wherein in step (3), primers used in the knockout verification are shown as follows:
Dnmt3b-Ex1PCR-L:
(SEQ ID NO: 3)
5′-GTGCCCCCATTTCTCCTACT-3′;
and
Dnmt3b-Ex1PCR-R:
(SEQ ID NO: 4)
5′-AGACCCAATGTGCTGGTCTC-3′.

8. A use of the Dnmt3b gene-deficient CHO cell line of claim 1 in the preparation of a target protein.


SEQUENCE LISTING (10 sequences)

SEQ ID NO: 1 - DNA, 25761 bp, Artificial Sequence
  synthetic
  misc_feature (17542)..(17566): n is a, c, g, or t
gaggccatagcaggtcagctgtcactcctggtgactgccagctctgtcactgcttttcctccatccttcttcccttcagaacttgggtttcacccccagctgatgaggggggaatgagtgacaggccaactaaccaatgggccccagaggatgggccggagtggggcttatgctgccatggagaagagagaagcctgcttgagaggaggggcagagggaaggagacctgaagagagagcaaacaaaggggtcacaaggggaaagagactggtttaaggtaaacagttgcttactcttcatttgctgctgcccagagacagaagggaacttaggctgtcccaagcaggcccaggcaagcatgtcctaaatggcatcatttgaagggctggccaattgcagagcagccctagctttagacctcagccttaacaccatcctctccctctgtcctggggcctggtatcagggacttggagattcacctaattagccccattgaaccaaatcctgagcctccaaatttggtgagcagacaggctattggtaggagtctagtgggaatacagacagacagtcagcttcagattgagtggggtctcttgggctggagatagagaacctgtaatatgaaacttggaatgtgcagcccttttgtgcccccatttctcctacttatcctgacttttttccaacaggaaacaatgaagggagatagcagacatctgaatgaggaggagggtgccagcgggtgtgaggaatgtctcatcgtcaatgggaactgtagtgaccaggcctcagatactaaggatgctccttcacccccagtcttggaggcaatgtgcacagaggcagtcaacacatcagagagcagaggtgggtgtcaaatctctgacatagcaggttccatggcaggtctcagtggagaccagcacattgggtctcttctgttgagttgggcttcatgatgatcaggagatgctgccaacttgcctcttcttagaagttagttccccgactgcctgaaagtccgcccccactggttgggggcctctatggtcccttctggtccttgacatctaccatgtcttttaggactttctgaaatggataaaagtggcctattttcctgttcaccccaagagctacttggccacacagggacatttgaaatgaagccagtatcaaccagtttcatggaatggactgtgaaggaatgggcttcttttcctccttagatgttaattaatgcttaacgtgtggagaggaaggcataagccaggccctgtcgctttgtccagtgagttcatggaggtgatgaggacactgattcctagaatgacagaatgttgaggatgcattttaaagcaaggccactccccaggctggtgatcactgtcctatttcccaaatgcagactcccaaatcttccttacaggccgaagatcaagctcacggctgtcaaagagggaggtctccaacctgctgagttacactcaggtaccatcatctcttttcttcatgtggttttataccgttcttttagttttgattgcatgagaaaaaggacaaatgaaattctttactccactgcaaagtcttttattcgatccttcccgtgtatttaaagttatgtctccctatctgtaatcagtattcaaatagaacaaaagacatttctggaatgacttcaccttggttgcggtatgtctgatttgcttgtttgagacagtatctggatggctgggatgtcactgtaagcaaacccctcgtctcgtgtcctctcacatgtgttgtgaatgagttgacgatgccggggaggcagcattctgcattagtctcatttccataatttgctggtgacttgggatttatcttttttttttttttaaagatttatttattatgtatagtgttctgtctgcatgtctgcctgcaggccagaagagggcaccagatctcataagggatggttgtgagccatcatgtggtttctgggaattgaactcaggccctctggaagagcagccagtgctcttaacctctgagccatttatccagatcctgggatttatcttttcaacccaatttgaatttgtgagcatgtgattagcaggagtgaaattttgctagagaaaaacctgatgatcattggtgtattttggactcctagggtaatgtcagtgcagaggtggtgatgtttctgtatactgattgcgttagttcgtcttttatccttaccataggacctggcaggagatggagatggagatggtgaagcagaggatggggatggctcagacattctactaatgccaaagctcacgcgtgagaccaaggagacaaggtcaccctcggaaagtccagctgtaagtccctcccacctggtaactctggcatctcccaccatgcctggacctggatagaaaataaggcttagacaattattcagaaaaaaatgtggacacatttggttttatgtgtgttgggtgtgtgtaggttatcatcttccagcatgtagattctggggacaaaactcaaggattggcaaggtttggcaagttccctaattcactggaccccttggttaaattttgatgttgcttagccacaacaggataattcagatccttacttgaacttgaaggtaaaaaaatgagagctgatagagcagatgagctttggacgtttgatacactgagcacagcaccaagagcttcatgaccactttacttttgattacattttgactttctgattacagtcccactgaggaaggctttgcaatcgtttgcttctcctgcttaaaactccaggaaactgcccacctcccatctttctctctttctctgtctgttactttccctctctgtctctctttccatctctctttgcttgtctctctgtctccctctcacctctctctgcctctctgcctcttttctctgctgctcctgtccacagaggctggtcttccctcttgcctcccttttcccatcccctttgccctaagaaaactctccacataaactttgttctatcgtgtctaatcctctttcgccactttttaaattacaacaatgaggagctgtattaaagagtcacagcgttaggagggttgagaactactggtttaaggtaaacagttgcttactcttcatttgctgctgccaaatcaggtggtgaaatctttgtgacatgggccagtgtgattgtccctttcaaagatacccttgttaaagggatccccacctttatatccaattatcttctccacttcaggttgactggaggctatctcagggcccaatccctttgggcaccttcattggaccccatgtccccagtcacctaaggccctgtgtttatcccttcttactttcaggttcgaacccgaaacagcaacattacctccagcctggagaggcaaagagcctcgcccagaatcaccagaggccgccagggccgccaccatgtgcaggaataccccgtggaattcccagctaccagggttggtcctgtctctgaaagatctgcttcactaatctgcacagcactggctataacctatgctttcttcacacacacctgcttttcagggtctggcatctttctttcctgactcatcatacttagaactactttgaggagtcaagagttccagattgcttaggaatctggcccaccttggagctgagctaaagccactggtgtctaccacgcatgtttcttttgttttttcctgctgtcttgaggcaaccccagcaggtccagcacattctacttctatttccagatgcacaggagccagtttcctggagaccccactctctctgagctcacagctcagagcctctggttgttacctgtcattgctctcacactatttattctgtttcctttctgcctttgggtcaaatggctgccaggtgcctgtccctgtaaccctgtcttctggggtgctggtttgctctttcactcttaacatctctgggaacctgttggtagacactgtacgtgtacctccttccactttgcttcactaggtctcagtcttgtctttctatctatagtctcggagaaggcgagcatcgtcttctacaagcacaccatggtcgtcccctgccagcccttatcccagtatcgacctcatagaagaagggacacctcagaggagcagtaccccatcaactgactggagccaggacagccagcaagagagtatggatgccacacaactgtatgcagagagcaaagatggagacagcactgagtaccaggtaccactgagaagggattttgtcccaggtgcacaagatccaaattacgggaaaagaaaacataaattatttattctcgatgggcgtggtggcgcacgcctttaatcccaggactgggaggcagaggcaggtggatttctgagttcaaggcctgccaggtctccagagcgagtaccaggataggctccaaagctacacagagaagccctgtctcaaaaaacaaaaaacaaacaaaaattatttattctccaaatgtcccctcacttccttttgtttttttgagatagcatttctttgtttaacagccttggctgttgtggaactcactttgtagaccagcctggcctcaaagtcacagaaatcctcctgtctctgcctcttcggtgccacatccaagtggtattttatccctgaaaaagatggtttcatagcatacataattaatcataaaacgcatgaggcagatctacagagcctgatctacagagaagatccgtgctatggctgtatagtgagactctgtctccaaacccaaagttttgtgtagccaaggatgacctccaaatccttgttctaccgaatgctgggaatatatgcatatgctactagactttctgttaattttactgggtataatccaagaacttccaaacacaatgtggccttttctttgttctaatccccacctttgttctaatcagttcagcattttagtagttccaattggacataatttttttggataatatttatgaccttagaaattttgatcctgttcaggcattacagtggcttttatcacttaagcaatagtttgctctcacactgtgtgtattcaggctctggagtgtatttaatgaattgagacttccttcctgcatgtggatgtatgcatctagacaaagcttcagagtaccttctgatggagtcctgtatgttgcttctttttttgtgaagcagtgttgtagttaaggttatttaggaggaagccattagcccaaggaacatgtgctcaagtgtgggcatgggcttctttatgctttcataatttcaggatggtgagctgactcagtggtaagagacacttggtaccaagcctcacctgagttcagtctctgggagaggacagacttctaacatgatgacctccacttgaacatacatgggttcctgcatacagccaaacatggacacacctctgcatacaaaaagttgtgaaaaaaatacatattattatacaacttatttttccttggcacctttttgtatgtaccattgagctatttctcaaactcatgtatcacattttatggccaacagtttttttcttgccctaggtcagtggagtttctcagggttcacctgccttggtgactgaaattctttctgttggttcttgcaggatgataaggagtttggaataggcgaccttgtatggggaaaaatcaagggcttctcctggtggcctgccatggtggtttcctggaaagccacctccaagcgccaggccatgcctggcatgcgatgggtacagtggtttggtgacggcaagttttctgaggtgagccatggcaaggtggtctcttctcctcttggggtagctctgaggtcagctttctgcatgaggatgcagtaagtttggcactgggactggagtaagaaaaatatatttcttggttacctgtcttagatgcctgcttgtaggaccagaaataagctgcttattctttttcttagagtagggtctctgtgtaaccctctgtacagtaggctggctttgaactctgaaatcggcctgcctctgcctcctgaggtgggggactcaaggtttgtgcagccacacctgtgacttgtgtactggagaataaggaagcagtatagcatttagagaggtagctgccatatctgcctgtcactccaatgccaaggttctaatgccctgctatggcctctgtgctcccccaccacacacacacacacacacacacacacacacacacacacaagtggtattgtgaaacatatcttacatacaccttgtctacacagcggagtccaggacagccagaggctgtataaagaaaccccatctcattaaaaataagagacagtatgagcttctaatgttaaacattttaaatgagtctttataatgatggtcttctcagcataaatgagagagtttattttctcccatcccatcagccccactttgactcttaactttaaacatgtgttctgagatctgctttaagatttttttaaatatgtatgcatcattctctatctatttctgtacaccatggaaggtcagaagagtattggattaaagtctctttattccaagcacagccgggtgaaacacaggccagagctaggttaagaacccagccagcgcagccagaacaaagagagagcgcggtccccacgtgcagccttttaagaagccccccttacgtcatgccggctttccttcaccccgcccttatgggcgagtccccaggtccacctggtacctgccccaggactattgggcggggctagggttactccctacagaagagtgatttcagatcttgactctggacgtcatgagctaccatctgggtactgggaatctaacccaagcctgtggaggagaaaccactgtactctttttgtttgtttgtttggtttttcgagacagggtttctctgtaggctgtcctggaattcactctgtagactaggctggccttgaactcacagaaatccacctgcctttacctcctgagtgctgggattaaaggcatgggccaccaataaccagcaaccactgtgctcttaaccacttggaattgtttctggtttggctttgagggctgcagaatggatttcctgggggtttccttagaaatgttagtcagtgttaggccaattcccagacacagacaggatggggtttcctgaagtacaacaaacaggtcggagtgaagtggcatttgggacttcgttccatttcaggttttctttcctatgagttggatttctatgacttttcctaaagtatatcctacatttccctgggtgacaacaaataggacttgaagactgttctgagaggttacatgcagttgccttgtctgaacagcagagggagcaggaggaatagtgacttgggtttatgcatcatttgccccatcttcttgccactcccttctctttggtatctctgccccaagccagtcttagaagaagggatatttgccattgctttgatccgaaagacaaaatatactcatctctggacagggtaagatggcaataagaggtagcatggagatggtagcattgtgggatagtgttagagtgtgccgtgatcctggcaaaaggcaaagacctgcccagggcctatatagcacccactgtgttctgctcatgttgggtagagcagctggcagttccttgtcacccagttttatctttgcactcactaggaatcccacccataattcatttctgcatcgacagaaggcaagttgcctttagtgattaggatataaggtatacaactattgcgactattaagaactcaagcatgcaaatcccagcacttgggaggcagaggcaggtggatctctgcgtttgaggcaagcctggtctacagagcgagtgccaggccagggtccaaagctacacggagaaaccctgtatcaaaaataaagaattcaagcatatgttcaacctgtgcacagtgggaaatccttggttggcagtgacaatctggaaggtaaggaacatgctcagatagattgtggacagtttcaggactgagactcaggtctgtaccagggacattcactctgttcatacacatgtatgtgcacatgtgcagggattttgaagacatacctagttttacttgaattatcctccttacccacatctggagtttctcatggccaaatttgctatatgccatttctcatttgccccttatttttcagttctggcatctgtaagttgttgcttcgttgggctctgggtgaaacagtgcacattaatctcaagtgtggttcgatcacggccatgtttttctctcctaggtctctgctgacaaactcgtggcccttgggctgttcagccagcactttaacctggccaccttcaataagctggtttcttataggaaggccatgtaccacactctggaggtaacatgggtgtgggtgctagctagggtgtcagcctataggcagggcagaaagaagaaacctatggagtaaaagcgagtagaaggtgtccctcaagactggcttggggcagagataccaaagagaagggagtggcaagaagatggggtctgacctctggttgggttgaactggttacaaggggaatcaccagtaagttgctaatactgatattaggtaagacaggaagattcatgacttgtgaggtcacctaatcacatttaagggccttttatcaatctgtgaacaggatttctaagcatagaggatgcaatggctagttttctcttctcatagtttgtagacagttgtttatccctgggaacctttgtgctgtggtggtaagtacatagatgtacagattctcttggccctggaaataatggttgcttggctacatcccttcagaagacatgagtcaaaggtagccttcctggataagctgttggcgatggggatggtcgtctcaatagctcagcttaatcaatctctgtcagggatctgaacatatctggtgcctacagggaaaaatgtctgtcacccatgtcccttactgttgtttccttacctgtccctcaaaagcagaccccttgtcacataacaccccaagacagctgtgcagctgtttcagacaccagtgtttaggtaatgcagtccccagcaggaggaatccctgggttcaccctggcacatacctcattcacctgctcccattcatggatagacttgatctttatttgtgtcttggcagagagccagggtgcgagctggcaagaccttccccagcagccctggagactcactggaggacaagctgaagcccatgctggagtgggcccacgggggcttcaagcccactggcatcgagggcctcaaaccgaataacaagcaaccaggtgggaatgaatctatacctctgttgatggtataggcctcagccacactggtaccctcagcactcttattgtgccaactcaagcaaggcctacttttactgtggaccccaggctgttaattaaaagcctctgggagcttgagggccattacctgccttgtctggcttccctggccatattgctgccatgggtgtgtgcagtgcaattaatcttttccacatatccaaattcatcttttacctttgtggccagattttctttgaaatgaaaaattaaaataaatttggctccctgccattccttccctgaatatgcgcaggattaacgtaatgaagtgaagggtgtccctgtacagccagctcctaatctaatggaatgtttttgttttgttttcctgcaaaagtgattaataagtcgaaggtgcgtcgtgcaggcagtagcaagttagaacccaggaaacacggtattcccttaccatctttgagtgttctgttttctttgctctgttctgcaatttcttggcatctacagtgctcactggagaagaatcaagtctcctaagctgatatgaatccctcccctcactccccgccctcaccgaccctcaggctaaccatgggtgcacatcccgcctcacacactctttggattatctccttctctcttgtttcttaacattaatgacatgtataagcactggaaattgtcactttgccatcaaaggatctttgaattttggtaactttaagtattttttttttgtgtgtgtgtgttggtgtttatgtgtggagatggagatcagaggacagggttcatgagtatagtctctaccgctggggtgactggtatcagattgctagacttaacctgcagacacgttgacccccagagctgtctcagtggccatgttttagtttcttgtgacagtgcctcactataaatcccaggctagatttggatacaggagtatgtcattacacatagcttccttatggagtttgatgggtttataattttatttccttgccatgccatgatggcacatgcctttaatcccagcacttaggagtcacaggcagacagagttcaaggccagcctggactatagagctgattcaaggacatccagggttataccaagaaaacctgtcctggaatacaaaaatttaatcaaatttaatcatttctaatatgagtcaatgtagaaacacgagaaagtgagccaaaataaaacatttgttttaaacaggttcttactatagccctcactgttctagaaccttctatgtagatttgggtggccttgaactttctgtctgtgctttctgaatgctgtcactaaaggaatgtactgtcacactcaggattaaaaaaatataacttaattttctagtaatagaacaactgaaatattaattatacatttaatctactttctaaattattacaaaattaaagtatttccatatttagtgatgtcttaagttgtatattggtgataaacatcacccaattgttaattccatattaactaatataaaattggctaaagtagattccattatttaagtcaagccagttgaaccaggcagtggcttcacgtgacgatttggacctgacccccagtgtgaaatcctgtccctcctgacatttcaaatagctccctgctgccattgagcattgtggtgggcatcactgtaggaacctcagagttccagctgtggcccagcatgtgctctgggctcactctagaaggatggattctcttacacattcagatgtgtaaaccccatgaacatgagtctggcctgcttcctgggtgagaggagctctgaattggcttctgctctgggctaccctctggggttctacacccaccccatcgtggcccctgctctagatgcagctggatcccaggccaagcatggtcttgtctgaggcccgctttgtctcttagaccttcagtcaaatgcgtctccttacctttcagagaacaaaagtcgaagaggcacaaccaacaacgactctgcagcttctgagtcctatccccctcccaagcgcctcaagaccaacagctacggtgggaaagaccgagggcaggatgatgagagccgaggtgatttggggtcgggtgctgaaaggcctggccatgcatggtgcataccaggagcagcaggtctgcagaggtgcatggctgcatgggaaaggagggacttccccacctcgtctcacaccttgtccctctttcctcaacagaacggatggcttctgatgtcaccaacaacaagggcaatctggaaggtactgttgtcccccggcacttccttctggggtgccccaggcctgtcttagcctggctttgaccttctgcatgggttcaatacagagcctgagcattgtcctacatcaggacctattcctgcacccatgtttaactgaatctgattttgtccgtgaagttactgaccagagagatcacagtaggggacaatgtgagctgcagcattttggccagcattggtaccttgtgctttccatgatctactttggtattgttccattatttttccttgcattcatttgtctcagaaccaagctattggagttttgaatatgtgagaaactccctgtttgccttcccaacttgcttctccttagtccttagagatagttgcttagtattgggtaagaaattttgctctgaacgtaggtaatgtctgtgagtaaagtgcttatgactcatataatacctgtgctgtggtcctgccaaaaccacgggaaaaaaaaaaaccaagctggccttgatgtgggttacactgactccctgcaatcccaggtcaaaaagacttaccctgtcctcttgttatgtgtgagcacagactgagtggatgtatacaagtaagtcgtttttaaaagcttggtttgtaccagtaacatggcccttttgagacagagatgggttagatataaggtttcatgatcatggtgcagggtgataatgcttcccatggaggggttaggacctcagattatttttccagaaatacagtaccacaaaggcaatgactaagccacctttgacagtcatggtgcttaagaaggaaattcaggacaggtcaagaaaagagaaactgccaggcagtggtgggcatgcctttaatcccagcacttgggaggtagaggcaggccatcctggtctacagatcgagttccaggacagcctccaaagccacagagaaactctgtctttaacaacaaaaaacatacaaactattttttaaaaaattttaatcctttttttgagaacttagttattatgtatataacattctgcttccatgtgtatctgcacaccagaagagggcaccagatccaattatagatggttgtgagccaccatgtggttgctgggaattgaactcaggacctctggaagagcagccggtgctcttaacctctgagtcatgtctccagcccctcaaaaaaccaatgttaaagaagagaattcctggacaaatggtatccagggacaagtgagatacattctgaccaatgttttagagtagaaagtcttaacacccatagtgttctcttgacctgtgtcatgagaatgtcttccttcaagaatatagtataaacatatacacacacacagacacacacacactctctctctctgtctcactcacagtgagtgggaaaaccaaacaactgtaagaaacccaaaatttaactatgctttccatagtttgcatatgtgcagctgtgcacacacatgtatataaaaataaagtataaaaatcatgggaaagttggtgtgcagaacaggctctcacagtatttgccttacctcccatgtagaagtcacccaacccctcctagtcagacagtttcggtcaggattattattatttatttctttaactatatagctatctgtcatctgtctatcctcctgtcattcctacctcgaatcttcaattagcaccagctgccgtagaattcactaagtagaccatattgtccaggagtactctatacctctgcttccagattcaggggtataaaggtcacatgaaaaacagatggattcagacctgaaattagacccaattagttaaaatctgctgattgttttaggtagaggagagagctggtgtctgaaggcacattaatgctacatcattggtaggatttgtagtgtcatctgcaaaacaaattgtggtttccattggtacaagaatttgtttggtgtttttctttgcaaaccccttgctcatgtgtagctcctttttgttgatttgaattagtaacatttaaattgaaacacatcattgcaaaagaacaaagtccttgacacagcaggatttcagaactctcacccttaaggcttcatcaggagatgttagccccttgatctccatagactctgacaccctgtctttctctcagaacgctgcttgtcctgtggtaggaagaaccctgtatccttccatcccctctttgagggtgggctctgtcagagttgccgggtaagtcttcctgccccttcctctgccacacccttccccagacaggcccccagcacagtccctgctccaggcgctaatgaacagcttcccagggcacatctcccaacacccttcctgtttggggacaggtgctgcagcagagaaggccctccctccataaggcactcagctcacatagactatcattgaagggcacccgggccaccagcttagggccccagacagcttgctagccagacctgactgaggccaggactatggtctcatgggctttctctctgactgtcaggatcgcttcctggagctgttctacatgtacgatgaggacggctatcagtcctactgcactgtgtgctgcgagggccgagagctgcttctgtgcagcaacacaagctgctgcaggtgagtgtccttctgtggggcctggacgagagggtctagggtccccgggtgtaccccaccgttcctgcagcctgtaggcagctaaaatccatagaaaatgagcacccagtggctattctgtgggctctggtgggcatgggcatggccttgggtcaggtgtgcctgtgagatcagccctgggaggtttgtgttgaaagggtctagatttgtagggagtgtgaaggacccacattcaagaagtggtttgttaccctatatctcaaagcatgtggaggcaggaggctgagcatcaggcaggggtggtcatgccctggggaactaactggacccgcagggcaaatgtctgctaaccgtgatcccgttctctctggatactctggcacaggtgcttctgtgtggagtgtctggaggtgctggtgggtacagggacagctgaggatgccaagctgcaggaaccctggagctgctatatgtgccttccacagcgctgccatggggtcctcaggcgcaggaaggattggaacatgcgcctgcaagacttcttcacttctgatcctgacctcgaagaattcgtaagccctatagagcctctgtttagtcccttgcaattgcagtccctgagaatctggattcctgtgcatctgtcaaagcaagagttaggcctttcatctcaggatgtaggagacaagcagtcatatggctgagttcaatgacagcctggtcagtcaagtgacttcccggccagtcaaggctacttatatactgagattctgtgtcaaaacagcccccaaaagttatagtaccaggcatagtgacagcacagatatctgagccatgagctttgtgatgagtttaagggcagcctgtgttaaatagtgaggtcaaggccagctgggaatgtgcagcaagaatccatttctactataccatttgtacctcacagagcccgtgccagaggctaagagcccttgctagtcaacacctctagcagacaattctcccttccagtaccttctgaggagggaacaggatggaaacatcccagcgtgagggtgacgagggcagctgctcccacaatgtaggagttgatcttctgttctcaggccttggccgttagcggtcaagctcctgatctggctctatgcgtctgtaggaggcagctgtgcctcctatctaaggacagaatggccagcctgtgcccagtgctctcttaccagttcccatggatgcttctgagtacaggaggatagatgcacactctgagacatcactctctttacttatctggcaggagccacccaagttgtacccagcaattcctgcagccaagaggagacctattagagtcctgtccctgtttgatggaattgcaacaggtgagttgaggcttatctgagcatgctctgttaggccaatggaagaatcttctagacgggctgaaggtgtctctcccatgataattgagaagagtagtagctaggatgtcatgtgtgtcctttcttaaacttttccttctatatgaataaataactagggacaattaggcaacagctgccttcctcaatcctggttccttccctttttgtgagctgtcccatacatagaaggaatgaacatagccaatactggcaatgttctgaggtccccggagtcgacacagacaccctgaagtgacagtgatgtagtaaggggttcttcagtgtgcttcaggaggacaccgtggctgggactgatggctctgcgcttggcctcttgtcctcacatttctgtttgatccctcacagggtacttggtgctcaaagagctgggtatcaaagtggaaaagtatgttgcgtccgaagtgtgtacagagtccattgctgtgggaactgttaagcatgaaggtcaaatcaaatatgtgaatgacgtcaggaaaatcacaaagaaaaatgtgagggtgacctttttcctggaattcatctcatcttgcttatttcttttctatacgctggaccttgccgagtctcaaggcatattggccctattcttcaggtccagggttgcaggtgtgagaagctgtggacacttcagaaaaaagtacaaatagggcagtttgagttatagctcaagattacttagttctgttagaacatgagcattcaggtgttaagcactgagtggtgagaagggtgatttgatgatttctttttctgtcttctcaacctctcctctcccctcccccagactccaaggttttagctcttccttccttaggttcatcctagctgtgtgtcttttcctcagattgaagagtggggcccttttgacttggtgattggtggaagcccttgcaatgacctctccaatgtcaatccagccaggaaaggcctatatggtgaggagaatgactggttttgtgaccccctccctgccagggccactatcagcagtgtcagttctgatggaatgggctgcaaagaaaatgtcggagtttctcaccttgctcatggcttaggccccattatattgtttggaattccagggcacaacgagtacaggctttattttattgccatttcctttggaaactgttggttttgggaaagttgaggcatatgctgatgaagctcctgattgaactctgttttccaatgctctcaatcaatatagagggcaccggcaggctcttctttgagttttaccacttgctgaattatacccgccccaaggagggcgacaaccgtccgttcttctggatgtttgagaatgtggtggccatgaaggttaacgacaagaaagacatctcccgattcctggcggtgagtggactgtcaggggagcctaggcagggaaggcactgccaacactgaaggctgcttgatactgctgaggaaatgattgcctgattagaggacggtatggctagatctacggatgaacagttcggcaaccttctaaacttaggtgatcctttggctcacctgtccttgttagctggcacttctcctatcttctgtttgtattgataatgactgggacagttactaagcttttgtctttccagttcttggtgtgttttctttcatttctaagctttattttgtatgtgtgcacacaggcacccctgccatgctgcatgtttgaaggtcacaggattactttggagtccattctctccacaatgtggcttctggatatcaaactctgggcatcagcataaaagcnnnnnnnnnnnnnnnnnnnnnnnnntgctgaaccatcctgctggctctgtgcttctttttgaagatggtctcacgtagccctggctttccttgaacctttactcttcctgtctctgcttctagagagcagggattacaggtgtgcaccaccacagctggcttataacttgtctagccttaagtgatagagctgtcctgaagccctggttaggcttgcatacattgcctgtacaaacttctgcagacataattttgacatgaatcagtttgggcaggtttgatcatcttgaccccaccatcttgcatagctctctccagccagcctctgggtgggacctggtcagtctcgttgcctcttctttagctaggaaaggcttgtcacccgacaggtcaaacatgggctgcctggcagctctagctaagtcttgaggtgtctctagcagctccctttctcttcttgcagtgtaacccagtgatgatcgatgccatcaaggtttctgctgcacatagggcccgatatttctggggcaacctacctgggatgaacaggtaagagacctgagagggacggcaaagtctgactgacatttcagttttctagaacagtggttttcaacctgtgggcaggacccctttggggcgggggaatccaatgaccctttcatagggttcccatatcaaatatttacattatgtttcataacagtagcaaaactacaattacgaggtagcagtgaaataatatgttggtcactacaactgttatttgggcttttttccattaatttatgcgtcctacatgcgtgcaggagcccatgaaagctatcaaccagtcctttgcttgagtggcaagcactcttaaccactgggccatctgtccagcccgaggaactgtattaaaggatcacagcattatgaaggttgagaatcactgtctagaaagaaggtcatggctttaggatgctctgaataactagactactggtcagctgtcttgctctagataaaggcggatatttctcaaggttctcatgatgactgagaactaacttcctgctgtgctaagtcttctattgcttccgagatcagacgagatcgggcgcgttctgtatggccgtagacaagtcttctattgcaaatgctgaatgtcaaagacatcatttttcctgaccctaaggtgaagttgtggtatgcatgtaatacatacagagaaaccattcataaaaataaaatcttttttccctcattctctatttaatccatctattatcttacttctctatagcaatatattctatatccatttgctttggattcctccatcttcttactgtatacctaaactatggagtgttgtagcatacacatatcccgagggcgtaaaagctattatgcaatataaagtagaacagacaatatttgacctttgtgtctggattatttcatgcaggatatttttcctggttcctgccatttcattattattatttttttttctttttaagacagggtctctgtgtagccctggctgtcctgaacctctgtagaccagactggccatggagatctacttacctctgcctcctgagtgctgggactaaaggcatggttcatcaccacccagctattttgtttttcttaacatctgaaaattccattgtatgaatgaatgacattttcactatccattaattgacagaacatctatgctgtttttgacttctctggcaattttgaatagatcagtaatgcacatgactgagcaagtggctctatagtagggtgtaggatgatatgtatacctttgggtacatcatcaagactggaataactgtatcttgaggcatatccatttccaacttcccaaggaactgccaaatggcacatagtgcctgaaccaatttgcatgcccaccagcaatgaataattgtttctttcccgttgcccacatccttgtcaccacgaggtatcatttgtattcatcttgtccattctgactgtcataagatgaaacctcggtaataatttgcatttgatttttctttttctttgagtctttttgtttttctttttttccccctttggacacagggttctgaaactctgtagaccaggttgccctcagattcacagagacctgtctgcctctgctgagattaaaaggcgcttgccaccgctgctcatctataattatactttcacagtgcctaggaatgttgaatattttattaagtctctctcaactgttcatatttcgtattttgaggagttagttcactacttctgatccccatttcttagttctttatagatactaaccctctattagatatatgattgctaagacattttcccattctataggctgcaattttgccacaagatgatgtccttacggccctatagaagttttatagaagtttttttttatttaaagatttatttatttatttatttatttattatatatacagtcttctgtctgcaggccagcagagtgcaccagatcttattacagccccctatagaagttttataagttgatggcatcccacgtaaaaattattgttcttagtgtctctttccacagaatctgatgccctcttgtcttctgagggtgacaggtatgcatgtgatatgcagataataaatgcagacaatactttcatacacataataaatacatttatttattataaaaatcatagactcggctttctaaccctaatcctagttttctcagacagtatttcctactctttaatcatgaaccaaaagttgtttagtcaagggagtttatagacaagcaatcgctctgtagaggcctgcttattcatgtttgctttcaagaccttgctaatgaatatagaaactttgatggtattgaccatctctgagggataactaagcagaggcatctggtgataaaccaatttggacattggccaagtgtcccaagaagggtaggcggagcctgtagtatggtgctccaggtgtcaggaggttatcattaaccacttaaccattgaagcttgctgaattactggtaccataccatgttcatttttgggataccctatgctgatttacatagcaatcaaactctctcattgctgttgtgttgcccattcaatggtggactctagtggtggcttttgtggagtggacaagctacttgagaaaggatagtaggtatgttcatggatgggaaggttgcttttcaaatgtggatgggaagaagaggtctcattcaacagaacaaaaggaatgcagctgtgtgaagaatgagcatcctatgagtgataaagggctctggatttcaggggttgatgggtttccctagacaagcttcaagatcagtgtttttaggtcaggaacaccttggagcgaaactaaattgcaggtttccatgtaagtaagcacagtaccggatacttattgttgtcattggaaaacatagggggtgagattgatttttgtatagtgagaggaaaagaggaagaaggcagaaagatacacaaaggaagtggtgtggtttattctctgtcaaggatgtcaacagctggagactcaagttatgtgacagccagcatgttggtcacaagaagagtgagcactgaagtctaggtggtggagacagacgtcactagctcatatctgttggattatcatccaccagtcaatctgagaattaaatttgtgcaaatccttatttttttaattatgcttatgtgtgtgcctgtgtgtgtgccttcatgctctatgaacacctgaaagctccatgttggtgtcacactccgctaaggggtccctggccttagacttgcccatgccccactacctgtgtggatgtcttgtgtggatccttgaatctgcacagatccaggcttcctctgacccttttacttggtctcaggtttagactcaattatttgtcattgggtggctagaagtctgtcaggtctggatgtcatttcttaattaaactgctttcattttgatgtctcagttattcggggcctataatggggctgctgtagttcatgacttctatgtacagatatggacaggtttctgctttgaaatccagctcatgatgtttgactccacatttgctgatcctatttagatggtcctatttagaaggagcaatcacattcattgcagggtggcatttactattactagactagccttctgagctcccattcctagccgtgcacgtctcatagtaaggatgtccgtggtgacctagaccaaggtgatagtgctgcctattgtaacagtggaggaacactttctgtttgcacaggacttggtcccctcttaatgcatgccattcagtaactgcaacactatacttcttgtgggtaatgggctgtttttaggcacttcagcataaccttgtgctctcagcaggatgtttttcatccaacatttgtgacttgatcattatctaagtacaattaggaagcagattctggagctgaggaactgtcagttcagagcacttgttgctcttggttcccttcccaacacctaaatggcagctcagaactgtgtaacctcagttctagaggctatactatacgttctcttcacttcctagaaccatgtctgcacatggtgttattacatatacaggcaaatgttcatacatagtttttaaattaaattttatttaaaaagctaggttctctcctcgtctgacttgcgaggtctctagcttagacctccagggcagatgctggagagtaacgggttttggctgttcccaggcctgtgatagcttccaagaatgataagctcgagctgcaggactgcttggagttcagtaggacagcaaaggtaagacgctgagtggtctatgctgcaccttctggaaaactgtgactttggctctatccacgtggctcccactcattccctcacagacccctggtgctgggcttctttccccacagaatagttctgtctcttcttcaagtacagagtaggagatgttcttggtaaataatccccgtgagacaatgtgaccgggctctgcagtggttaatgctggaggcatagaggctgtgctgcagggagtctttgcagaggcaacagtggcagacgctattgcaaaaggctatgtgctgtgcagtcagagatgcactgtaaggcgtccggtgcctgctcctccatgtctgtgaaaagcaaaggcctattgctcaagctctggttgggcattctgagtctggatggtttttgctcttaatgacccctttggtcaataactggtgtcttgggcaccatgtgatccttcatggtcacttttggctaaaatgtatgtggcatttgtggagagcatatagaatctcttcaagtgtctttgtcctctctctctttggatgcctgtccctgccacctctccaaactcttgccttctgtcctctgccctctggatgcccctgagcctgtctctacttggaagccttctgtgctcacccggcgatgttgttttgttctccagttaaagaaagtacagacaataaccaccaagtcgaactccatcagacaggggaaaaaccagcttttccctgtagtcatgaatggcaaggacgatgttttgtggtgtactgagctcgaaaggtgagctgcacgtggagagggaacccttgggaacaaaggcaggaagggggaaatgaagcaagagaggacagtggacacctgtctgtattccctgatatttccctgtcccagaatctgctttaaatgagaaagctatggaaactctgcctttatccccagatttctgctccaatgggtacttttctccctagtcttccttgagcaatcatctctttcattcagttgtgcagttttttgttttggttttgttttgggtctggctggcctggagctgaatatgtatatggccatgaattcagaaagatcctcctgccttttttatttatttatttagcttcttgttatagggtttctctgtgtagccctggctatcctggaacaagctctgtagaccaggctggccttgaacgcacaaacatccttctgcctctgcttcctgagtgctgggattaatggcgtgcaccaccaccacctgtcctcttttccagatattttatattaattatttcatgaagatgttaatatttcatatttcattacatgtttcttttctaatctcctggccgagtatggattcactatagtctagacacttctgtgctaaagttggccttaaaaatggccttaaaaatttcttttaccaggctggagagatggctcagaggctaagaacactcattgctcttctaggggtcctgagttcaattccacatggtggctcacaaccatctggaatgagatctggtgccccctcttctggccttcaggcacacatgtagacagaacactgtatacataataaataaatccaatcaattagttcttttggttttttgagacagggtttctctgtggctttggaggctgtcctggaactagctcttgtagaccaggctggtctcaaactcacagagatccgcctgcctctgcctcccgagtgttgggattaaaggcgtgcgccaccaacgcccggctccaatgaattcttttttaaatttgagcatatttgcctcttccctaatactgggattcttacttccaccactatatggtgggatcttgcattggtctggtgcataccatcacaatactgtgtgttcatatgtgcttaagccctgttaagtctgaaaacagtttcagtatattatcaacttcttcaggctcttagcagtctcagtgccctcttctgcaaagctccctgggccataggctgaggtcacttattatgtccatgctgtatagcctgttctgaaatttgaaagtttaaagtggttcttgaggtgtgattggtactgtgtagatgccaggaatagcatggtctggccttgaggtgttgaattcctacaggtaactttagagctgtcaaaagccagcgatactgcggttcagtctttgtcctcttcagcatcctgatgaagcacattaggcaagctctggatttttctctcatattttgagagagagcaagagtactgtgtatgtacatgtgctcaactggctatggaagaactggtatcttgtaagcccctgtgtttgtcattgaaaatcttgtcttttgtttcttcccctggaaaacaaatgctttgactgcttattatgttttgaagccaatgttcacactatgcatttaagaatggtcctggatacatgtctgctgactggtgatcattcatgggttaatttacactaggcatttaagaagcatccttgcatggcatgtaaggagcacagtcccagtgaagatacacagcactggttccctgtcactgtcagctgttagctgagctaacaattgaactggtggtctagaaggttcctttgagcccagtggtcctgactgaagcctcatggctttcctaggcctggacttcgaaaacacagctgttctggtgtgctggggacatgggcccttctaaccttgtgtcttttcgctctgttctctcccaggatcttcggctttcctgcacactacacggatgtatccaacatgggccgtggtgcccgccagaagctgctgggaaggtcctggagtgtgccagtcatcagacacctctttgcccccttgaaggactactttgcatgtgaatagttctacccaggactggggtacttttggtctgagccaggtccccagagtcacccctccctgaaggcacctcacctctcccgtttctcagctcacctgtttggggccccaactcactgtgtacctcagctttctcctgctcagtgggagcagagcctcctggcccttacaggggagccaaggtgctcccaccatgtgcacaacttagaactggctgcttagagtagcccaatatggtgctcatgttttcttatcctgaaactttaaaacttgaagtagatagtaagatggcttttttcccctcttgggttaatcagtcagaagtgatggctaagataccaaaacgacggctctcccagtactcagggtaatgctgcaaaatcacttgattttctttttaaataacctatttccacatttgctagaggatgccaatggcaatgtgggctcagatgaacaaggtcaaggggccagaattttttcaaaaatgttttccctaggga

SEQ ID NO: 2 - DNA, 288 bp, Artificial Sequence
  synthetic
gtgcccccatttctcctacttatcctgacttttttccaacaggaaacaatgaagggagatagcagacatctgaatgaggaggagggtgccagcgggtgtgaggaatgtctcatcgtcaatgggaactgtagtgaccaggcctcagatactaaggatgctccttcacccccagtcttggaggcaatgtgcacagaggcagtcaacacatcagagagcagaggtgggtgtcaaatctctgacatagcaggttccatggcaggtctcagtggagaccagcacattgggtct

SEQ ID NO: 3 - DNA, 20 bp, Artificial Sequence
  synthetic
gtgcccccatttctcctact

SEQ ID NO: 4 - DNA, 20 bp, Artificial Sequence
  synthetic
agacccaatgtgctggtctc

SEQ ID NO: 5 - DNA, 23 bp, Artificial Sequence
  synthetic
gaggaatgtctcatcgtcaatgg

SEQ ID NO: 6 - DNA, 23 bp, Artificial Sequence
  synthetic
cttggaggcaatgtgcacagagg

SEQ ID NO: 7 - DNA, 27 bp, Artificial Sequence
  synthetic
caccgaggaatgtctcatcgtcaatgg

SEQ ID NO: 8 - DNA, 27 bp, Artificial Sequence
  synthetic
aaacccattgacgatgagacattcctc

SEQ ID NO: 9 - DNA, 28 bp, Artificial Sequence
  synthetic
caccgcttggaggcaatgtgcacagagg

SEQ ID NO: 10 - DNA, 28 bp, Artificial Sequence
  synthetic
aaaccctctgtgcacattgcctccaagc